(19) United States
(12) Patent Application Publication
(10) Pub. No.: US 2010/0137711 A1
(43) Pub. Date: Jun. 3, 2010

(54) Apparatus and Method of Analyzing Arterial Plaque

(75) Inventors: James A. Hamilton, Newton, MA (US); Alkystis Phinikaridou, Boston, MA (US)

Correspondence Address: Sunstein Kann Murphy & Timbers LLP, 125 SUMMER STREET, BOSTON, MA, 02110-1618, US

(73) Assignee: TRUSTEES OF BOSTON UNIVERSITY, Boston, MA (US)

(21) Appl. No.: 12/625,923

(22) Filed: Nov. 25, 2009

Related U.S. Application Data
(60) Provisional application No. 61/117,911, filed on Nov. 25, 2008.

(51) Int. Cl. A61B 6/00

(52) U.S. Cl. 600/431

(57) ABSTRACT

A method of identifying arterial plaque analyzes arterial plaque using one or more non-invasive tests to determine if the plaque has any of a plurality of hallmarks that are predictive of disruption. The one or more tests do, in fact, test the plaque for the plurality of the hallmarks. The method then formulates a vulnerability quantity as a function of the determined hallmarks. The vulnerability quantity identifies whether the plaque is vulnerable to disruption.

### CROSS REFERENCE TO RELATED APPLICATIONS

The present application claims priority from U.S. Provisional Patent Application Ser. No. 61/117,911, filed Nov. 25, 2008, which is hereby incorporated by reference herein in its entirety.

This invention was made with Government Support under Contract No. HL061825 and HL083801 awarded by the National Institutes of Health. The Government has certain rights in the invention.

### TECHNICAL FIELD

The present invention relates to analyzing arterial plaque.

### BACKGROUND ART

Atherosclerosis is known as a chronic disease that can progress for years without symptoms, and then spontaneously result in an acute ischemic event caused by plaque rupture or erosion and thrombosis. In spite of diagnostic, surgical, and therapeutic advances in the treatment of subjects with coronary and carotid atherosclerosis, vascular disease and its subsequent ischemic complications, including myocardial infarction (MI) and stroke, remain among the most important cause of morbidity and mortality in the developed world. The incidence of vascular disease is increasing proportionately with increases in obesity and type 2 diabetes mellitus in the population. In the U.S., atherosclerotic burden and the prevalence of coronary heart disease is greatly increased when type II diabetes is present. A recent meta-analysis (June 2006) of 37 prospective cohort studies showed that the rate of fatal MI was higher in persons with type II diabetes. Clearly, early diagnosis of atherosclerosis before the additional and potentially irreversible damage that may occur because of plaque rupture is an increasingly important priority.

Atherosclerosis is a complex disease with multiple factors contributing to initiating events, plaque maturation, and plaque rupture. It is characterized by accumulation of lipid, calcium phosphates, inflammatory cells, and proteoglycan in the subendothelial matrix of the arterial wall. These components mainly affect the intima, but secondary changes also occur in the media and adventitia. In addition, micro-vessels within the plaque have been found in plaques with vulnerable features, mainly by histology. A large body of work has established that accumulation of low-density lipoprotein is a major source of plaques lipids, primarily cholesteryl esters (CE) and cholesterol. The importance of intra-plaque hemorrhage and erythrocytes as contributors to plaque rupturing has been shown in human coronary arteries.

A major milestone in atherosclerosis research was the discovery that inflammation and a deregulated immune response contribute to both the chronic (plaque progression) and acute aspects (plaque rupture). Moreover, there is considerable evidence that the plaque rupture/erosion, which precedes most incidences of stroke and MI, does not occur in stenotic lesions that cause severe luminal narrowing. Of the coronary lesions that cause death, approximately 70% are ruptured plaques, and most of these are non-stenotic. While considerable research has focused on the complex biochemical, immunological, and signaling aspects of atherosclerotic development, there is also renewed interest in the ultra structure of atherosclerotic plaques. As judged from histology, plaques that have disrupted and have thrombus formation at the site of rupture (a process termed atherothrombosis) are characterized by a lipid-rich core and a thin, fibrous cap. There are multiple factors that contribute to plaque rupture, and additional characteristics of the arterial plaque, such as infiltration with inflammatory cells, are also considered strong predictors of plaque vulnerability.

Many factors dispose individuals to atherosclerosis, but conventional risk factors account for only about 50% of known cases. Biomarkers in blood for lipid abnormalities and low levels of systemic inflammation do not specifically predict or localize the underlying pathology. Even with the revised and lowered target of total plasma cholesterol of 200 mg/dl, half of the acute events occur in subjects with <200 mg/dl. While changes in lifestyle and diet and widespread use of statins have decreased the overall incidence of cardiovascular disease and are effective therapeutics for subjects after a non-fatal event, the prediction of sudden death has seen little or no progress.

There are presently no reliable blood biomarkers known to the inventors' for the prediction of high-risk plaques. Additionally, the discovery of a reliable blood biomarker would still prove problematic in determining anatomical location or severity of the atherosclerotic plaque. Accordingly, providing a safe and rapid method for site-specific identification of high-risk plaque would be a significant improvement in the current state of the art.

Because most plaques that rupture or erode and result in ischemic complications do not produce a flow-limiting stenosis, they are difficult to detect by conventional methods prior to an acute event. MRI has the potential for identifying plaques that are likely to rupture/erode, and for following plaque progression or regression. Numerous MRI studies of atherosclerosis in the human carotid artery, both in vivo and ex vivo (with and without contrast reagents), have detailed plaque components by signal intensity using different imaging sequences. Plaque imaging by MRI remains a basic research tool with great potential but without very limited clinical applications at present. The promise of in vivo imaging at low field (1.5 T) for determining some features of plaque ultra structure was shown in early studies. MR images of human carotids with sufficient resolution were obtained from patients treated with lipid-lowering agents and controls that allowed segmentation of four individual plaque constituents. Morrisett et al. correlated T1 and T2 relaxation values with composition in different regions of the images of carotid plaques, and showed that MRI could detect changes in carotid atherosclerosis in vivo at 16 and 24 months in patients receiving statin therapy. Similarly, MRI showed that aggressive lipid lowering therapy by statins decreased carotid plaque volume in human subjects.

Another aggressively pursued MRI approach is targeted imaging with particles, sometimes “nanoparticles” that bind to the endothelium. The goal is to find new reagents that bind with higher affinity or are taken up into plaques that are higher risk. One limitation of this approach is that should such a targeting particle be developed, it will need extensive toxicity testing and is not readily available.

### SUMMARY OF THE INVENTION

In accordance with various embodiments of the invention, a method of identifying arterial plaque analyzes arterial plaque using one or more non-invasive tests to determine if the plaque has any of a plurality of hallmarks that are predictive of disruption. The one or more tests do, in fact, test the plaque for the plurality of the hallmarks. The method then formulates a vulnerability quantity as a function of the determined hallmarks. The vulnerability quantity identifies whether the plaque is vulnerable to disruption.

The plaque may take the form of a plurality of plaque sites along a blood vessel. Thus, in some embodiments, the method images the blood vessel to locate the plurality of plaque sites. In that case, each plaque site has an independently determined vulnerability quantity.

Illustratively, the method analyzes the section of an artery using a plurality of different tests. To that end, the method may image a section of a blood vessel after exposure of that section to a contrast agent, and then quantify contrast agent absorption of the imaged section. In addition, the method may calculate a remodeling ratio of an artery having the plaque and quantify the heterogeneity of an artery having the plaque.

Various embodiments analyze the plaque with a pre-specified sequence of hallmark determining tests that may be performed in a single imaging session. For example, the method may execute the following acts, in the following order:

A. quantify the heterogeneity of a portion of an artery having the plaque,

B. determine remodeling of the portion of the artery, and then

C. analyze the circumferential rim of the portion of the artery.

Some embodiments also convert the vulnerability quantity into a rupture percentage, which indicates the likelihood of rupture of the plaque.

In accordance with another embodiment of the invention, an apparatus for identifying arterial plaque has an imaging device for non-invasively imaging a blood vessel, and an analysis module (operatively coupled with the imaging device) configured to analyze arterial plaque imaged by the imaging device. The analysis module uses one or more tests to determine if the plaque has any of a plurality of hallmarks that are predictive of disruption. The one or more tests are configured to detect the plurality of the hallmarks, if present; i.e., if one, more than one but not all, or all of the hallmarks are present, the test(s) will detect them. The apparatus also has a processing module (operatively coupled with the analysis module) for formulating a vulnerability quantity as a function of the determined hallmarks. As with other embodiments, the vulnerability quantity identifies whether the plaque is vulnerable to disruption.

In accordance with another embodiment, a method and apparatus for identifying arterial plaque receives data, from an instrumentality, relating to arterial plaque retrieved using one or more non-invasive tests. The method and apparatus respectively use this data to determine if the plaque has any of a plurality of hallmarks that are predictive of disruption. The one or more tests do, in fact, test the plaque for the plurality of the hallmarks. The method and apparatus also formulate the above noted vulnerability quantity as a function of the determined hallmarks.

Illustrative embodiments of the invention are implemented at least in part as a computer program product having a computer usable medium with computer readable program code thereon. The computer readable code may be read and utilized by a computer system in accordance with conventional processes.

### BRIEF DESCRIPTION OF THE DRAWINGS

The foregoing features of the invention will be more readily understood by reference to the following detailed description, taken with reference to the accompanying drawings, in which:

FIG. 1 schematically shows a system for analyzing plaque sites in accordance with an embodiment of the present invention.

FIG. 2 schematically shows one implementation of the embodiment shown in FIG. 1.

FIG. 3 shows a process for analyzing plaque sites in accordance with an embodiment of the present invention.

FIGS. 4A-4B illustrate a heterogeneous plaque that ruptured.

FIGS. 4C-4D illustrate a homogenous plaque that did not rupture.

FIG. 5 illustrates a signal intensity comparison of the standard deviations of stable plaques and ruptured plaques.

FIG. 6 shows a statistical comparison of stable and vulnerable plaques characterized by homogeneous, intermediate, and heterogeneous compositions in accordance with an embodiment of the present invention.

FIG. 7 shows an assessment of negative and positive remodeling in accordance with an embodiment of the present invention.

FIG. 8 shows a statistical comparison of stable and vulnerable plaques characterized by negative, positive, and intermediate remodeling in accordance with an embodiment of the present invention

FIGS. 9A and 9B illustrate examples of a reference vessel in accordance with an embodiment of the present invention.

FIGS. 9C and 9D illustrate negative remodeling in a stable plaque in accordance with an embodiment of the present invention.

FIGS. 9E and 9F illustrate positive remodeling in a vulnerable plaque in accordance with an embodiment of the present invention.

FIGS. 10A-10I show stable and vulnerable plaques imaged before, during and after exposure to a Gd-DTPA (gadolinium) contrast agent, and corresponding histology.

FIG. 11 shows a statistical comparison of stable and vulnerable plaques with and without contrast agent enhancement.

FIG. 12 shows a testing sequence and timeline of arterial plaque analysis in accordance with an embodiment of the present invention.

FIG. 13 shows a comparison of a vulnerable and stable plaque analyzed in accordance with the testing sequence and timeline of FIG. 12.

### DETAILED DESCRIPTION OF SPECIFIC EMBODIMENTS

In illustrative embodiments, an apparatus and method analyzes arterial plaque sites to determine how likely those sites are to disrupt; i.e., they determine the vulnerability of the plaque site to disrupt. To that end, the apparatus and method quantify this likelihood based on the number and/or quality of disruption predictive hallmarks of the plaque site. This quantification may be represented by a numerically specified quantity (e.g., a percentage of likelihood of rupture), or expressed in a range, such as in the form of a confidence interval. Details of various embodiments are discussed below.

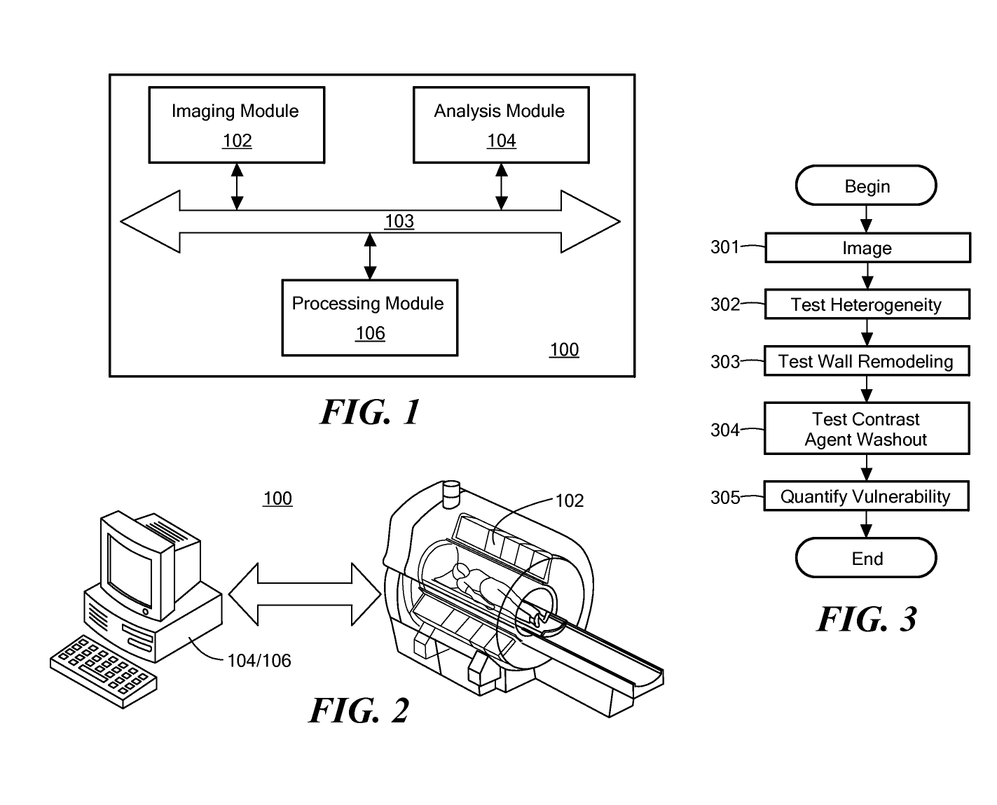

FIG. 1 schematically shows a plaque identification system 100 configured in accordance with illustrative embodiments of the invention. As noted above, this system 100 images and determines the likelihood of rupture of one or a plurality of plaque sites. For example, the system may image an artery with plaque. That artery may have a plurality of separate plaque sites that each should be analyzed to determine if it is vulnerable to rupture and thus, potentially cause a catastrophic event within the living being (e.g., within a human being).

To those ends, the identification system 100 has an imaging device 102 for non-invasively imaging relevant internal systems, such as a patient's vasculature, and an analysis module 104 configured to detect an analyze plaque within the patient. A processing module, operatively coupled with both the imaging device 102 and analysis module 104 via a bus 108, processes data received from the analysis module to determine if the located plaque is vulnerable to disruption. These three primary components thus cooperate to determine plaque vulnerability.

It should be noted, however, that additional components may be included with in the system 100—the system 100 merely is a simplified schematic of a larger apparatus that accomplishes the desired goals. For example, the system 100 may also include a monitor for displaying results, or additional processing modules. In a similar manner, the functionality of individual components may be modified or combined into different configurations. For example, the processing module can incorporate the functionality of the analysis module (or vice-versa), thus leaving the system 100 with two components. In that case, the single processing module effectively forms the analysis module and processing module. Those skilled in the art therefore should understand that discussion of the specific components is illustrative and not intended to limit various other embodiments.

In accordance with an embodiment of the invention, the processing module generates a vulnerability quantity, which may be in the form of a percentage or a number based on a scaled score. This quantity indicates the likelihood that a plaque site will experience a rupture; i.e., it indicates degree to which the plaque is vulnerable to disruption. Some embodiments express this vulnerability quantity as a percentage likelihood of rupture. To that end, the vulnerability quantity may be further processed, such as by using a look-up table or calculation, to produce such a percentage. Alternatively, the vulnerability quantity itself may be expressed as a percentage.

FIG. 2 schematically shows one implementation of the embodiment shown in FIG. 1. Specifically, FIG. 2 shows the system 100 as being formed by a magnetic resonance imaging device implementing the functionality of the imaging device 100, and a personal computer implementing the functionality of the analysis module 104 and processing module 106. Accordingly, in this embodiment, software modules within the computer perform the functionality of the analysis and processing modules 104 and 106. Interactive software and user interfaces enable a user to select and specify criteria, which may control the analysis of arterial plaque. For example, the software may prompt the display of an image captured by the imaging device 102, and request that the user make a selection based on the image before the analysis proceeds. The selection may include outlining or selecting a specific region for analysis, selecting one of a plurality of plaque sites for analysis, or both.

Although the imaging apparatus shown in FIG. 2 is a magnetic resonance imaging (MRI) apparatus, such a device merely is an illustrative embodiment of an imaging apparatus for imaging a subject (e.g., a person or animal). Of course, various embodiments are not limited to such a device. Accordingly, instead of or in addition to an MRI apparatus, various embodiments of the present invention may use other non-invasive imaging devices, such as (among other things) X-ray machines, computed tomography, fluoroscopy, angiography, and ultrasound imaging. Furthermore, some embodiments of the present invention may include the use of more than one form of imaging apparatus.

FIG. 3 shows a process for analyzing plaque sites in accordance with illustrative embodiments of the invention. The process begins at phase 301, in which the imaging device images at least a portion of an artery. This portion of the artery includes a plaque site or region within the artery where there is at least some plaque accumulation within the wall of the artery. Once the required image or images are obtained, the system 100 may begin testing. Specifically, the system 100 may execute a series of tests to determine if the artery has one or more hallmarks indicative of plaque rupture.

More specifically, the inventors understand and discovered that a plaque site vulnerable to rupture may have any of a plurality of different hallmarks. While having any one hallmark does not necessarily imply a high likelihood of rupture, it can indicate caution or a possible concern. During testing, however, the inventors discovered that plaque with multiple hallmarks can indicate a high likelihood of rupture. Thus, the process undertakes a testing regime (e.g. one or more tests) to test for some plurality of hallmarks. Three such hallmarks are discussed.

The first test applied is the heterogeneity test, step 302. The test for heterogeneity, which will be described further detail below, analyzes a plaque site based on the variation of biophysical components of the site. The variation in the components may be characterized based on the variation in signal intensity exhibited by the site as demonstrated by an image of the site.

This test may be followed by step 303, which characterizes wall remodeling of the test site. Wall remodeling is described in greater detail below. In summary, however, wall remodeling generally relates to 1) an outward expansion of the outer wall of an artery at a plaque site, referred to as positive or outward remodeling, or 2) a decrease in the vessel area and a corresponding contraction of the lumen, referred to as negative or inward remodeling. The test may include determining a remodeling ratio, which may be obtained based on the vessel area and/or outward wall circumference of a reference site. The test for remodeling may be achieved through analyzing a cross-sectional image of the artery or vessel wall, where the outer vessel wall is identifiable. Once these walls are identified at a plaque site, the circumference or diameter of each may be measured either manually or through a fully or semi-automated process. The vessel area then may be compared to a reference site to characterize any remodeling that may have occurred. The characterization may specify whether there is negative or positive remodeling, and it may specify the extent of any remodeling by providing a remodeling ratio.

The wall remodeling test demonstrated in step 303 may be followed by a contrast agent wash-in or absorption test (step 304). A contrast agent may include, but is not limited to, gadolinium. While gadolinium may be used for imaging, this use is still considered to be non-invasive in accordance with various embodiments of the invention. The level of wash-in or absorption of the contrast agent may be characterized based on a cross-sectional image of an artery at a plaque site. The characterization may specify the degree to which the vessel wall maintains the contrast agent after a specified time period. The degree to which the contrast agent is obtained within the vessel wall may be indicated by a percentage or ratio of the vessel wall circumference that demonstrates the contrasts agent therein after the requisite time. Accordingly, the image required for such a test may be required to be obtained after the lapse of a specified period and one or more images may be time stamped for comparisons.

The test at steps 302-304 thus identify 3 hallmarks of vulnerability (to rupture). Specifically, heterogeneity, remodeling, and contrast agent wash-in, discussed below. Of course, other tests may determine these or other hallmarks. Accordingly, discussion of these specific test are illustrative and not limiting of other embodiments.

In one embodiment, the hallmark may simply be a binary quantity (either a “one” or a “zero”) identifying whether or not the hallmark exists. So, for example, the site may have two of the three tested hallmarks. Other embodiments may not have binary results. Various embodiments of the present invention require that the totality of characterizations test for the existence or non-existence of more than one hallmark, such that the quantification provided is representative of a characterization of more than one hallmark. The characterization may specify that the hallmark does not exist at the site.

Step 305 thus generates a quantity identifying the likelihood of rupture. The quantity may take the form of a rupture percentage. In some embodiments, the quantification that occurs in step 305 may be based on the characterization of only two tests, for example, the heterogeneity test and the wall remodeling test. Additionally, while these tests are in a specific order in some embodiments of the invention, other embodiments may alter this order, and or substitute one or more of the tests above for an alternative hallmark identifier. A hallmark includes, but is not limited to, wall remodeling, wall heterogeneity, and contrast agent wash-in or absorption. Specifically, as discussed in greater detail below, the remodeling ratio, particularly outward remodeling, is often indicative of some likelihood of plaque rupture. The other hallmarks that are also discussed in greater detail below, such as little contrast agent wash-in, indicated by enhancement or a high degree of contrast agent remaining in the vessel wall and high levels of heterogeneity also have a predictive value of plaque rupture. The existence or non-existence of such a hallmark, which may be determined based on some threshold of the characterization, may be used to quantify the vulnerability of the plaque site.

As noted above, the existence of a hallmark may be characterized by a binary quantity, e.g., a zero indicating that the hallmark is not present at the plaque site and a one indicating that the hallmark is present at the plaque site. Accordingly, the vulnerability quantity may be a culmination of such binaries, so that the existence of three hallmarks produces a vulnerability quantity of three. Alternatively, a logical AND or a logical OR may be applied to the hallmarks to arrive at the vulnerability quantity. For example, in various embodiments, the vulnerability quantity may be a culmination of all the hallmarks, while in other embodiments the vulnerability quantity may be a culmination of one or more of the hallmarks. Additionally, various embodiments may provide a vulnerability quantity that is the culmination of two specific tests, which may be a set testing configuration, or may be adaptable based on the initial analysis results prior to quantification.

In some embodiments, each hallmark is equally weighted and hence affects the score in equal proportions. In other embodiments, the vulnerability quantity may be determined based on unequally weighted hallmarks, where the existence of certain hallmarks or certain combinations of hallmarks have a more significant impact or influence on the overall quantity than other hallmarks or other combinations of hallmarks.

In yet other embodiments, non-binary quantities may identify the hallmark of a given test. For example, in a remodeling test, if the expansion is within a first range, then the output for that hallmark may be a first quantity, while if the expansion is within a second range, then the output for that hallmark is a second, different quantity. The output of that test thus is either the first or second quantity, which is used with the output of the other test(s) to arrive at a final vulnerability quantity.

Rabbit models of atherothrombosis have been performed to verify the validity of various embodiments of the invention. Human atherothrombosis and plaque rupture or erosion (plaque disruption) cannot always be studied in a controlled manner, necessitating animal models to confirm the ability of the current invention to validate predictions of plaque disruption. The New Zealand white (CNZW) rabbit model of atherothrombosis is an established model of controlled atherothrombosis. Detailed studies by conventional methods of dissection, and examination by light microscopy, histochemical staining, and electron microscopy have established some similarities between plaque rupture in this animal model and in the human, although some features of human pathology were not replicated in the CNZW rabbit.

This animal model permits triggering of plaque disruption and the opportunity to detect thrombus formation and to discriminate features of vulnerable and non-vulnerable plaque. Studies of the invention focused on plaque disruption and the distinction between non-disrupted and disrupted plaques by MRI with validation by histology. The research shows that after controlled triggering of plaque disruption, thrombus may be visualized by MRI within hours. Molecular targeted MRIs were used to enhance the detection of the thrombus associated with plaque disruption by use of a fibrin-targeted peptide (EP-2104R).

The dietary protocol for the CNZW studies was modified and has shown that the aortic plaques produced by this modified model encompass almost all of the eight categories specified by the American Heart Association (AHA) for humans. The modified CNZW model includes the essential features of plaque rupture and erosion in humans. The rabbit model allowed the development and testing of various embodiments of the invention. However, the applications of some embodiments of the invention are not limited to the rabbit and may be extended to humans.

In accordance with one embodiment of the present invention, in vivo MR experiments were performed under deep sedation using a 3T Philips Intera Scanner and a synergy knee coil with 6 elements in which the rabbits were placed supine. A pulse oximeter was placed on the animal's ear for cardiac gating. The upper and lower abdominal aorta of all atherosclerotic rabbits was imaged twice; before (pre) and 48 hours after (post) the first pharmacological triggering. Control rabbits were imaged once. A saggital, 3D, phase contrast MR angiogram (PC-MRA) was acquired with a repetition time (TR)=20 ms, echo time (TE)=3.5 ms, flip angle=15°, number of excitations (NEX)=2, slices=25, slice thickness=1 mm, flow velocity=75 cm/s, matrix (MTX)=256×244 reconstructed to 512×512 (in-plane resolution=586×586 gm) and scan time=3 minutes. Then 2D, T1-weighted, axial images (4 mm) of the aorta were acquired with a black-blood (BB), double inversion recovery, turbo spin echo (TSE) sequence and cardiac gating. Imaging parameters included: inversion time (TI)=350 ms, TR=2 cardiac cycles, TE=10 ms, TSE=15, NEX=2, slices=25, MTX=384×362 reconstructed to 512×512 (in-plane resolution=234×234 gm), scan time=7 minutes. Immediately after a bolus injection of Gd-DTPA (0.1 mmol/kg, IV) (Magnevist, Berlin, Germany) a 3D, PC-MRA with axial slices was performed. For every axial, T1BB slice (4 mm) a total of 8×0.5 mm slices were acquired using a TR=17 ms, TE=7.4 ms, flip angle=15°, NEX=2, slices=200, flow velocity=75 cm/s, MTX=128×122 reconstructed to 256×256 (in-plane resolution=0.195×0.195 μm) and scan time=8 minutes. Contrast-enhanced T1BB images were repeated 10 minutes after the injection of Gd-DTPA (0.1 mmol/kg, IV) (Magnevist, Berlin, Germany) as described above.

As noted above, three measures may be used to predict plaque vulnerability to rupture in accordance with an illustrative embodiment of the present invention. All the MRI predictions were validated by a functional definition of plaque disruption in the live rabbit by a second set of images after triggering for disruption of vulnerable plaques. The MRI was then validated by histology.

### Plaque Heterogeneity

The presence of multiple chemical components and cell types is known to be a characteristic of complicated plaques, which results in an overall more heterogeneous MR signal. The variations in signal intensity reflect the different relaxation properties of individual protons species, which arise from their mobility differences and the differences in the chemical environments. Without knowing the precise plaque constituents that contribute to the heterogeneous MRI signal, the inventors used the overall plaque heterogeneity as an indicator of plaque complexity. The visually apparent plaque area was outlined on T1BB images. The standard (SD) of the mean signal intensity of the pixels comprising the plaque was measured.

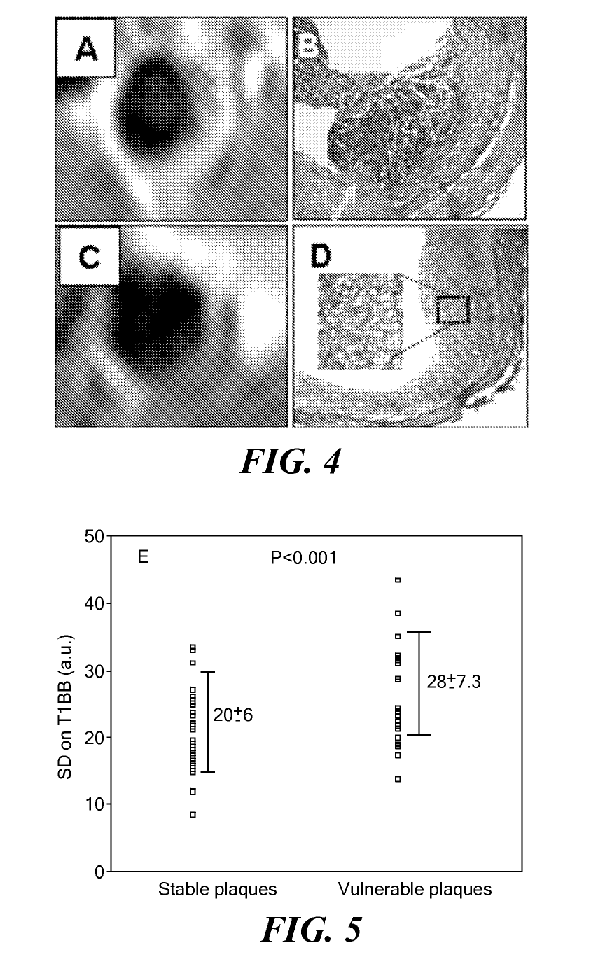

FIGS. 4A-4B illustrate a heterogeneous plaque that ruptured and FIGS. 4C-4D illustrate a homogenous plaque that did not rupture. As demonstrated in the figures, the inventors noticed that vulnerable plaques appeared more heterogeneous on T1BB MR images (FIGS. 4A-4B) compared to stable plaques (FIGS. 4C-4D). The corresponding histology of a representative vulnerable plaque showed that it consisted of foamy macrophages, collagen fibers and necrotic core (FIG. 4B) while the stable plaque was mainly comprised of foamy macrophages (FIG. 4D).

FIG. 5 illustrates a signal intensity comparison of the standard deviations of a stable plaques and plaques vulnerable plaques. When the SD of the mean pixel intensity comprising the plaque was used as an indicator of plaque heterogeneity, vulnerable plaques had statistically higher SD of the pixel intensity compared to stable plaques (28±7.3 versus 20±6, P<0.001) (FIG. 5).

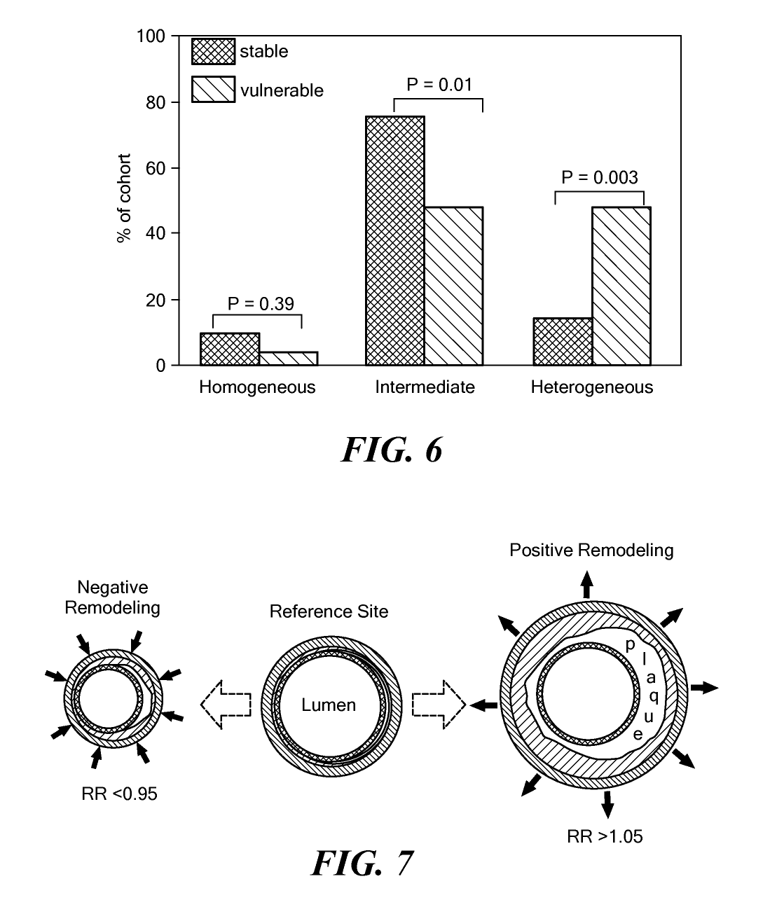

FIG. 6 shows a statistical comparison of stable and vulnerable plaques characterized by homogeneous, intermediate and heterogeneous compositions in accordance with an embodiment of the present invention. As shown in FIG. 6 the frequency of vulnerable plaques with heterogeneous appearance was also higher compared to stable plaques (48 versus 14.6%, P=0.003). In contrast, stable plaques had higher frequency of intermediate appearance (75.6 versus 48%, P=0.01). There was no statistical significance between the homogenous appearance in stable and vulnerable plaques (4 versus 9.7%, P=0.39).

### Positive Remodeling

Positive remodeling is highly predictive of vulnerable plaques. Angiographic studies, which image only the lumen of the vessel, suggest that the majority of vulnerable plaques cause <50% luminal narrowing. This is possibly due to the presence of compensatory enlargement or positive/outward remodeling that has been reported in histological studies in both humans and animal models. Positive remodeling is not visible on angiographic images. Positive remodeling involves the expansion of the vessel area as a response to plaque growth. Although positive remodeling is initially advantageous, since it alleviates luminal narrowing, histological studies also suggest that it is associated with increased expression of matrix metalloproteinases (MMPs) associated with plaque rupture. In contrast, inward or negative remodeling refers to shrinkage of the vessel wall. Although it causes more luminal narrowing, negative remodeling has been associated with plaques that are more stable and may produce symptoms that give a warning of vascular disease.

Arterial remodeling has been confirmed in vivo in patients with coronary atherosclerosis using high-frequency epicardial echocardiography, http://circ.ahajournals.org/cgi/content/full/95/7/1791—R5 intravascular ultrasound (IVUS) and in vivo MRI. However, suggestions of an in vivo correlation between positive remodeling and plaque vulnerability have been mainly drawn from IVUS, an invasive method.

In the inventors' study, the pre-contrast enhanced (CE) T1BB images were used to calculate the plaque area (PA) and the % cross-sectional narrowing (CSN) by manually segmenting the adventitial and luminal contours of the vessel wall. Plaque area was calculated as: PA=adventitial area−lumen area and the CSN as % CSN=(plaque area/vessel area)*100. Un-gated 3D PC-MRA images acquired immediately after injection of Gd-DTPA were used to calculate the remodeling ratio (RR) and the % stenosis from flow-compensated/anatomical and flow-encoded images, respectively. In the anatomical images, (T1-weighted spoiled-gradient echo) flowing blood appears bright whereas the contrast of stationary tissues depends on the T1 relation times. In flow-encoded images, only flowing spins elicit signal, and the intensity is proportional to the velocity of flow, whereas stationary tissues are suppressed. It has been shown that spoiled-gradient echo images detect the adventitia/outer region of the vessel wall and that the delineation of this contour becomes improved in contrast-enhanced images. Thus, at each lesion site, the anatomical images were used to measure the vessel area (VA) for the calculation of the RR, and the corresponding flow-encoded images were used to calculate the unobstructed lumen area (LA) and the % stenosis.

FIG. 7 shows an assessment of negative and positive remodeling in accordance with an embodiment of the present invention. The arterial remodeling ratio and the percent (%) of stenosis were calculated after correcting for arterial tapering and inter-individual variability of arterial size. The RR was calculated as RR=vessel area lesion/vessel area reference and the three remodeling categories were defined as previously described: positive if RR>1.05, intermediate if 0.95<RR<1.05 and negative if RR<0.95. The percent (%) stenosis was calculated as: percent (%) stenosis=1−[lumen area lesion/lumen area reference]*100. Because of diffuse vessel wall thickening, the slice with the least amount of plaque was used as a reference site, assuming that it was least affected by the disease (mean values of references: PA=2.0±0.56 mm², VA=11.0±3.5 mm², LA=7.2±1.5 mm² and percent (%) CSN=21.4±6.3).

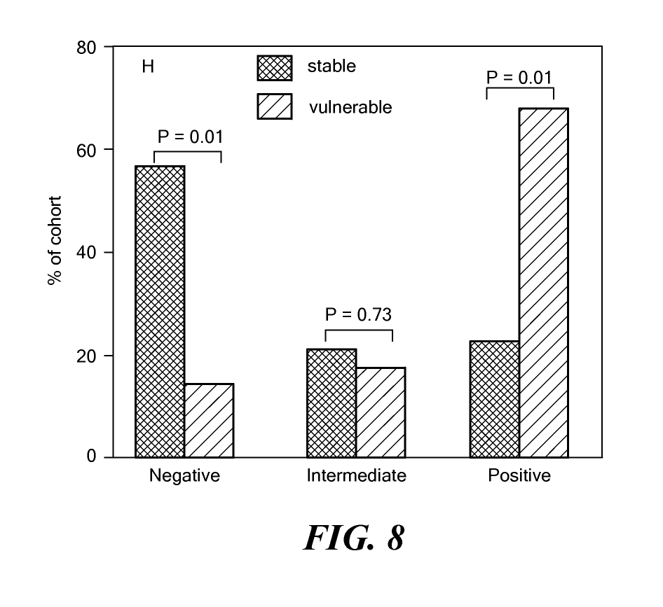

FIG. 8 shows a statistical comparison of stable and vulnerable plaques characterized by negative, positive, and intermediate remodeling in accordance with an embodiment of the present invention. In FIG. 8, the remodeling category is shown on the horizontal axis and the frequency of each category in stable and vulnerable plaques in shown on the vertical axis. Negative remodeling was significantly greater in stable plaques while positive remodeling was significantly greater in vulnerable plaques. Intermediate remodeling was similar in the two groups.

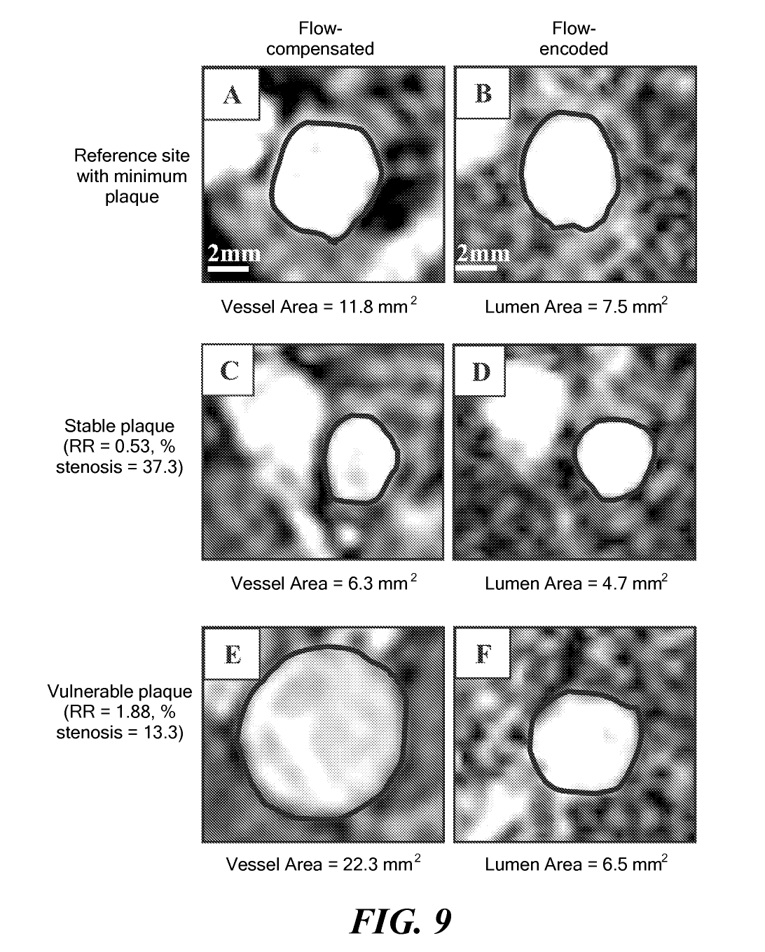

FIGS. 9A-9F illustrates representative Pre-triggered PC-MRA images acquired from the same rabbit demonstrate examples of negative and positive remodeling compared to a reference site. The vessel area measured at the site of the stable plaque (FIG. 9C) was smaller than that of the reference site (FIG. 9A), which is indicative of negative remodeling. In contrast, the vessel area of the vulnerable plaque (FIG. 9E) was markedly larger than the reference site, suggestive of positive remodeling. Images of the lumen (FIGS. 9B, 9D, 9F) demonstrate that this example of a stable plaque exhibited a greater extent of stenosis compared to that calculated for the vulnerable plaque. Positive remodeling was more common in vulnerable plaques (67.8% versus 22.3, P=0.01, sensitivity=67.8, CI=0.38-0.78). Conversely, negative remodeling was statistically greater in stable plaques (56.7% versus 14.2%, P=0.01, specificity=77.6, CI=0.75-0.95).

### Contrast Enhanced MRI

Vulnerable plaques show circumferential enhancement after administration of Gd-DTPA. Contrast enhanced MRI (CE-MRI) using gadolinium-diethylenetriamine penta-acetic acid (Gd-DTPA) has been reported to improve the discrimination between the fibrous cap and the lipid core and the visualization of coronary atherosclerosis. In addition, dynamic CE-MRI showed that the uptake of Gd-DTPA is correlated with neovascularization and inflammation both of which are increased in vulnerable plaques. Previous studies showed increased uptake of Gd-DTPA in inflamed plaques due to increased neovascularization and endothelial permeability. These changes result in increased wash-in kinetics as well as in regions of tissue necrosis due to increased distribution volume and decreased washout kinetics. Yuan et al. who reported that increased delayed contrast enhancement was associated with plaque severity.

In the inventors' study, the T1BB images acquired after administration of Gd-DTPA were visually compared to the baseline T1BB images to evaluate the presence or absence of a visually apparent circumferential or crescent-shape enhancement pattern of the vessel wall at the lesions site. Bright signal was sometimes visible around the outside of the vessel wall (adventitia) on the baseline T1BB images. These regions represent perivascular lymphatics and/or fat. To eliminate ambiguities regarding the presence of the enhancement pattern on Gd-DTPA-enhanced images, these regions where outlined on the baseline T1BB and subsequently masked onto the contrast-enhanced images.

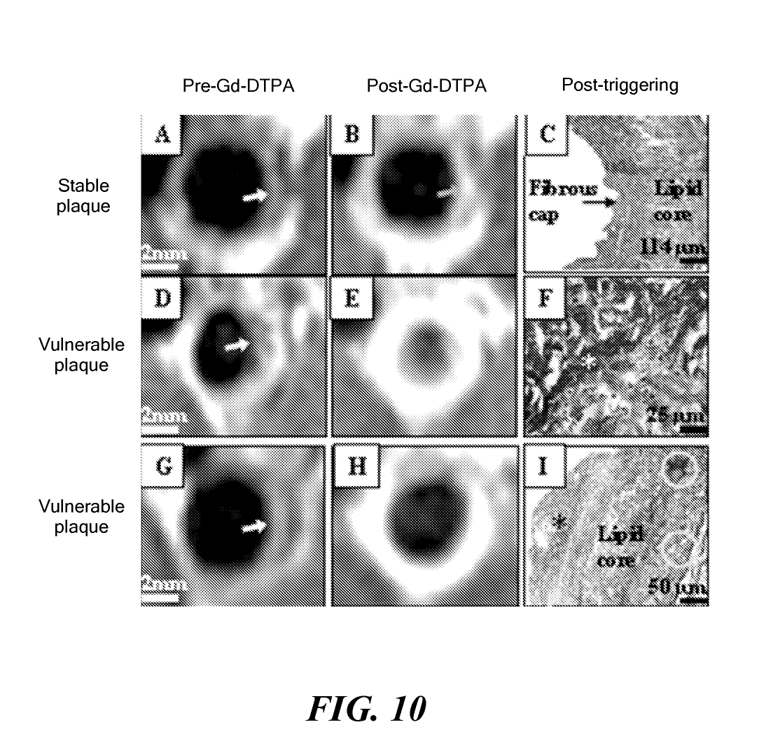

FIGS. 10A-10I show stable and vulnerable plaques imaged before, and after exposure to a Gd-DTPA (gadolinium) contrast agent. The Figs. represent pre-triggered T1BB images of stable and vulnerable plaques acquired with and without Gd-DTPA together with histological sections collected after pharmacological triggering. FIG. 10A shows a small, stable plaque which exhibited overall mild enhancement after administration of Gd-DTPA (FIG. 10B). A focal region of higher contrast-enhancement is also seen within the plaque. Histology verifies the presence of a stable plaque with a thick fibrous cap overlaying a lipid core (FIG. 10C). In contrast, the vulnerable plaque illustrated in FIG. 10D demonstrated strong circumferential enhancements with Gd-DTPA (FIG. 10E). The corresponding histological section (FIG. 10F) confirmed that a thrombus formed after pharmacological triggering. The extensive red blood cells (RBC) filled neovessels (yellow arrow) that span the fibrous cap may explain the increased uptake of Gd-DTPA on the pre-triggered images. Another example of a vulnerable plaque is illustrated in FIG. 10G. Unlike the vulnerable plaque shown in FIG. 10E, this plaque demonstrated a crescent-shape enhancement after administration of Gd-DTPA (FIG. 10H). Histology (FIG. 10I) verified the presence of a thrombus, which overlaid an extremely thin fibrous cap (yellow arrow). In addition, several RBC filled neovessels where seen within the intima and the adventitia of this plaque (yellow circles).

FIGS. 10A, 10D, 10G show MR images before triggering and FIGS. 10B, 10E, 10H) after injection of Gd-DTPA. FIGS. 10C, 10F, 10I show corresponding histology after pharmacological triggering. FIG. 10B demonstrates stable plaque with mild enhancement. A focal region of higher enhancement (green arrow) is also seen. FIG. 10C demonstrates Masson's trichrome and shows a thick fibrous cap overlaying a lipid core. FIG. 10E demonstrates vulnerable plaque with circumferential ring enhancement and FIG. 10F illustrates Masson's trichrome and verifies the presence of a thrombus. Note that red blood cells filled neovessels (yellow arrow) and span the fibrous cap in this Fig. FIG. 10H demonstrates vulnerable plaque with a crescent enhancement. FIG. 10I demonstrates Masson's trichrome and shows a thrombus (asterisk) attached at the luminal site of the thin fibrous cap. The yellow circles indicate neovessels in the intima and adventitia.

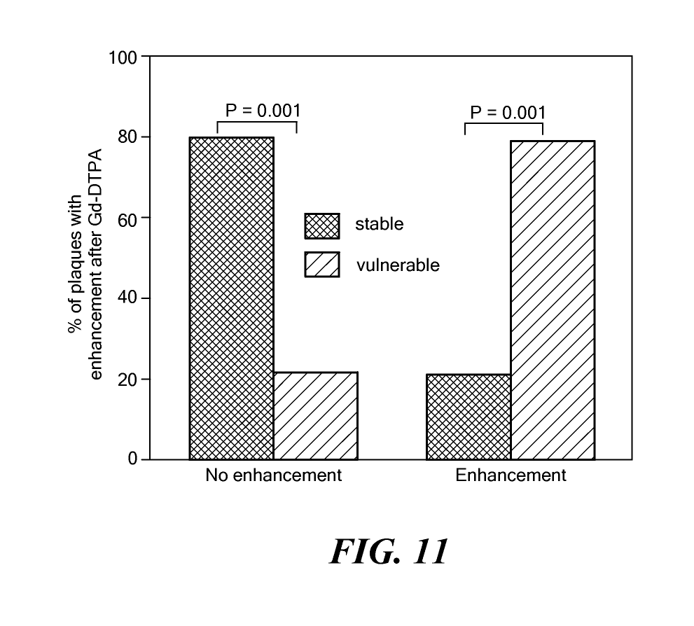

FIG. 11 shows a statistical comparison of stable and vulnerable plaques with and without contrast agent enhancement. The frequency of the circumferential or crescent-shape enhancement was statistically higher in vulnerable compared to stable plaques (78.6% versus 20.9%, P<0.001, sensitivity=78.5%, CI=0.63-0.95). Conversely, the absence of enhancement was statistically higher in stable than vulnerable plaques (79.1 versus 21.4%, P<0.001, specificity=79.1%, CI=0.65-0.91) (FIG. 11). Contrast enhancement of the vessel wall was not observed in neither the uninjured nor the injured control rabbits (data not shown).

When both the RR and the Gd-enhancement tests were combined to detect vulnerable plaques, the sensitivity of the test was 50% and specificity increased to 97.0%. Multi-logistic regression analysis identified the gadolinium hyper-enhancement (P=0.01, Odds ratio=13.46, 95% CI=3.17-57) and increased vessel area (P=0.004, OR=1.36, 95% CI=1.1-1.68) as independent predictors of plaque vulnerability.

As demonstrated in FIG. 11 and as noted above, the frequency of the presence of circumferential and crescent-shape enhancement pattern after injection of Gd-DTPA in stable and vulnerable plaques was statistically higher in vulnerable compared to stable plaques. In an embodiment of the present invention, the characteristics of this circumferential and crescent-shape enhancement pattern after injection of Gd-DTPA may be evaluated and quantified. In addition to or as an alternative to evaluating the intensity of the Gd-DTPA around the plaques, a risk assessment may be provided based on the amount of the perimeter of the vessel wall that is covered. For example, a threshold may be set at 70-80%, and an observation that 70-80% of the circumference or perimeter of the vessel is covered may qualify the site as a high-risk or vulnerable site.

The MRI method contains three components for prediction of plaque stability/plaque disruption. All three have been validated in a rabbit model that functionally defines “vulnerable/high-risk” plaques. All three are based on structural, compositional and biophysical differences between sable and unstable plaques. Additionally, the methods have various advantages, some of which include: assessments without requiring the use of x-rays, compatibility with current human imaging systems at low field (3T), rapid processing time of 1 hour or less, multiple imaging protocols with a powerful and predictive approach generally not achieved with a single protocol test, and compatibility with approved contrast agents.

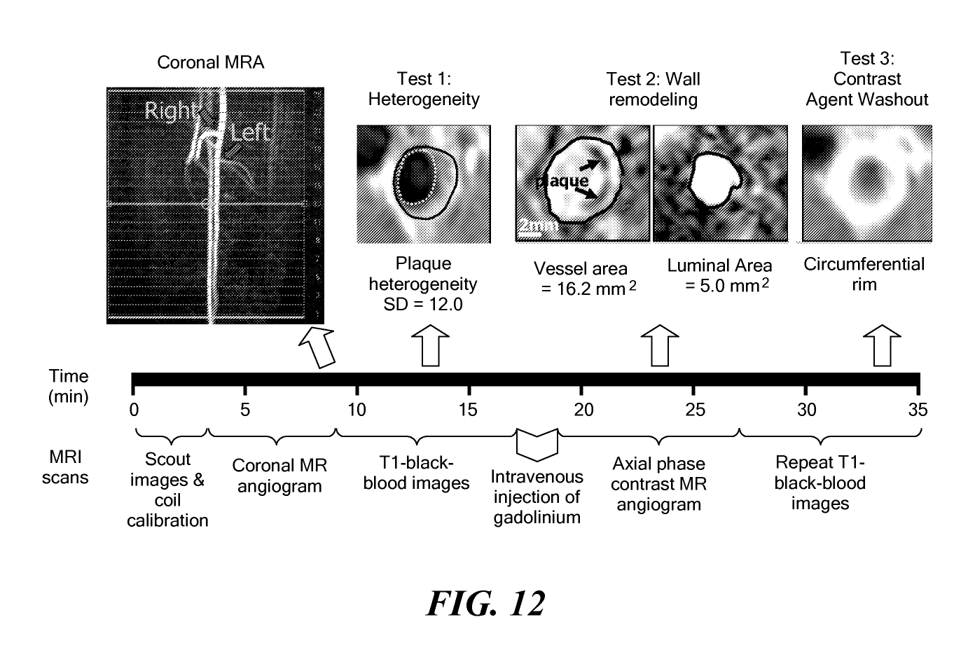

FIG. 12 shows an exemplary testing sequence and timeline of an arterial plaque analysis in accordance with an embodiment of the present invention. Following the production of this image is a first test for plaque heterogeneity based on T1-black-blood images. The image allows the vessel wall area to be distinguished and also allows the variation in the signal intensity of the vessel wall area to be analyzed. Based on the variation in the signal intensity in this region, the composition of the wall area may be analyzed to determine if it is heterogeneous or homogeneous, and the extent of either as specified by the standard deviation of the signal intensity within the region. This test may be conducted between 10 and 15 minutes into the diagnostic analysis.

In this exemplary embodiment, a contrast agent, in this example gadolinium, is intravenously injected into the subject between 15 and 20 minutes after the beginning of the analysis. The second test for wall remodeling may be engaged after injection of the contrast agent. The cross sectional images for this test may be obtained between 20 and 25 minutes after initiation of the analysis. The test requires that the vessel area, bounded by the outer vessel wall, be compared to the luminal area, bounded by the inner vessel wall. Wall remodeling may be characterized with respect to a reference vessel area indicative of a normal vessel wall area.

After the contrast agent is taken up into the vessel wall, the third test may be conducted. The amount of contrast agent maintained within the vessel wall may be characterized after the wall has had ample time to absorb the agent into the wall and has had ample time to wash the agent out of the wall through the permeability of blood into the wall. Once this has had time to occur as referenced by the process in a reference location, a cross sectional image may be obtained to determine what portion of the vessel wall has not removed the contrast agent from therein. This image may be obtained between 30 and 35 minutes in the illustrated embodiment. Accordingly, this embodiment of the invention requires only about 35 minutes worth of imaging time.

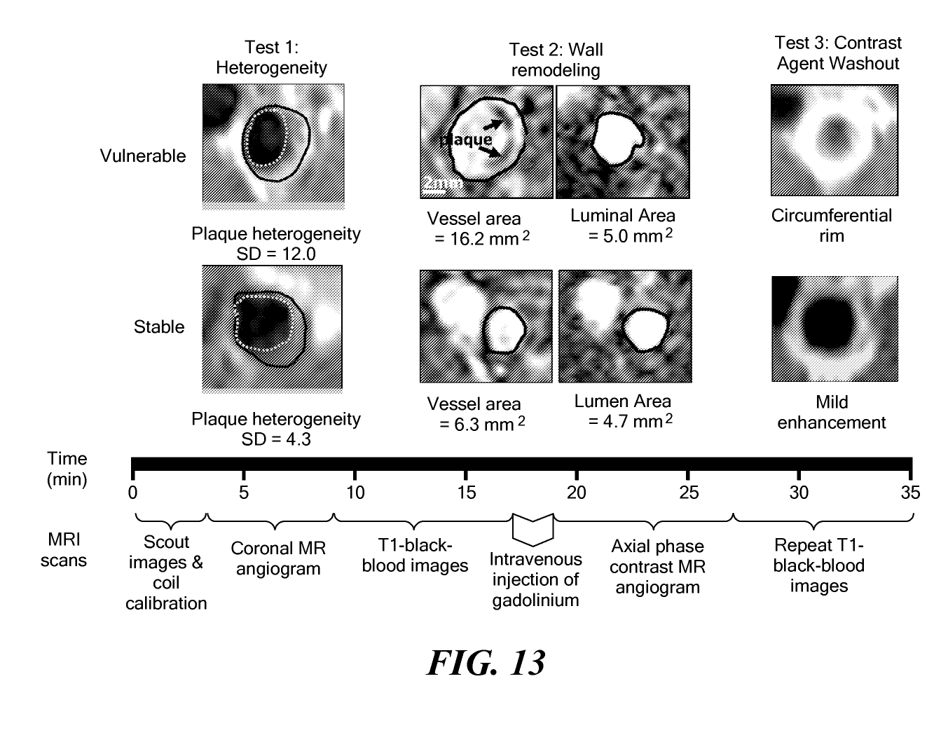

FIG. 13 shows a comparison of a vulnerable and stable plaque analyzed in accordance with the testing sequence and timeline outlined and described with reference to FIG. 12. As demonstrated by this Fig., which depicts a retrospective comparison completed during research, the plaque heterogeneity of a vulnerable plaque that ruptured exhibited a standard deviation of 12, while a plaque that remained stable and did not rupture exhibited a standard deviation of 4.3 (variation in signal intensity based on plaque composition). Additionally, the ruptured plaque site, before rupturing, had a vessel area of 16.2 mm² and a luminal area of 5 mm². In contrast, the non-ruptured plaque (stable) had a vessel area of 6.3 mm² and a luminal area of 4.7 mm². The ruptured plaque also exhibited a complete circumferential rim before rupture, while the non-ruptured plaque (stable) exhibited only mild enhancement of the contrast agent, hence substantial washout of the contrast agent at the same point in time.

Based on one an analysis of 100 rabbit aortic plaques, the sensitivity and specificity of each of these analyses was determined retrospectively alone and in combination. The sensitivity indicates the percentage of the subjects with vulnerable plaques that were correctly identified, while specificity indicates the percentage of the subjects without vulnerable plaques that were correctly identified. The test for heterogeneity was shown to exhibit 42.8% sensitivity and 76.1% specificity. The test for wall remodeling was shown to exhibit 67.8% sensitivity and 77.610% specificity. The test for contrast agent wash-in was shown to exhibit 78.5% sensitivity and 79.1% specificity. The sensitivity and specificity of these tests in various combination was also calculated. A combination of all three tests was shown to have a sensitivity of 21.4% and a specificity of 100%.

Table 1 shows the positive predictive value, negative predictive value and the diagnostic accuracy of the same analyzed subjects based on the three tests alone or in some combination. The results demonstrate the basis on which a computer program in accordance with embodiments of the present invention might be programmed to use certain combinations of test indicative of certain hallmarks to quantify the vulnerability of a plaque site within a subject based on the availability of the identified hallmarks.

TABLE 1

| | | | Positive | Negative | |
|---|---|---|---|---|---|
| | | | predictive | predictive | Diagnostic |
| | Sensitivity % | Specificity % | value % | value % | Accuracy % |
| Positive remodeling | 67.8 | 77.6 | 55.9 | 85.2 | 74.7 |
| Gd-enhancement | 78.5 | 79.1 | 61.1 | 89.8 | 78.9 |
| Plaque Heterogeneity | 42.8 | 76.1 | 42.8 | 76.1 | 66.3 |
| Positive remodeling or Gd- | 96.4 | 59.7 | 50.0 | 97.6 | 70.5 |
| enhancement | | | | | |
| Positive remodeling | 50.0 | 97.0 | 87.5 | 82.3 | 83.2 |
| and Gd-enhancement | | | | | |
| Plaque Heterogeneity or | 85.7 | 58.2 | 46.1 | 90.7 | 66.3 |
| positive remodeling | | | | | |
| Plaque Heterogeneity and | 25.0 | 95.5 | 70.0 | 75.3 | 74.7 |
| positive remodeling | | | | | |
| Plaque Heterogeneity or | 82.1 | 58.2 | 45.0 | 88.6 | 65.2 |
| Gd-enhancement | | | | | |
| Plaque Heterogeneity and | 39.3 | 97.0 | 84.6 | 79.2 | 80.0 |
| Gd-enhancement | | | | | |
| Plaque Heterogeneity or | 96.4 | 43.2 | 41.5 | 96.6 | 58.9 |
| positive remodeling or Gd- | | | | | |
| enhancement | | | | | |
| Plaque Heterogeneity and | 21.4 | 100 | 100 | 75.3 | 76.8 |
| positive remodeling and | | | | | |
| Gd-enhancement | | | | | |

The analysis methods described contain, in some embodiments, three components/hallmarks/tests for prediction of plaque stability/plaque disruption. All three components have been validated in a rabbit model as discussed above and used to define “vulnerable/high-risk” plaques functionally. All three components are based on structural, compositional and biophysical differences between stable and unstable plaques. Additionally, the methods of various embodiments of the present invention have a number of advantages such as: assessments without the use of x-rays, compatibility with current human imaging systems at low field (3T), rapid processing time of 1 hour or less, multiple imaging protocols with a powerful and predictive approach not generally achieved with a single protocol test, and compatibility with approved contrast agents.

The discussed analysis methods enable use of a scoring system that assesses the risk of arterial plaque rupture in a subject. The score may be provided on a site-by-site basis. Site-by-site assessments also may be used to score the subject as a whole. Such a score may be termed a “vulnerability score” and may be calculated automatically by a computational system through assessments of specified parameters. Such a score provides a tool for clinicians and patients to discuss the patient's state of health and possible remedies to prevent or limit the risk of death or injury upon the occurrence of a triggering event. As noted above, the score may be a function of one or more assessments made with regard to, among other things, absorption of a contrast agent, measurement of a remodeling ratio, and measurement of plaque heterogeneity. For example, the intensity of the contrast agent, as detected through MRI, may affect the score such that for given incremental increases in the contrast agent, intensity level the patient's score may be incrementally increased. Additionally or alternatively, a patient's score may be adjusted up or down, depending on the amount of perimeter coverage achieved by the contrast agent (as determined from the image). The score may also be increased if the remodeling ratio is above a specified value. Threshold levels may be specified such that heterogeneity levels may be deemed low, medium, or high and the heterogeneity level measured may also alter the score of a patient. The level of each parameter, contrast absorption, remodeling ratio and heterogeneity, may be specified and programmed into a computational system that measures these parameters and calculates a corresponding score. Additionally, the levels of the parameters may be adjusted as needed based on age, past medical history, or other relevant factors, such that a proper assessment and score is provided for the specific patient.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented, at least in part, as preprogrammed hardware elements (e.g., application specific integrated circuits, FPGAs, and digital signal processors), or other related components.

In an alternative embodiment, the disclosed apparatus and methods (e.g., see the flow chart described above) may be implemented as a computer program product for use with a computer system. Such implementation may include a series of computer instructions fixed either on a tangible medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink-wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of identifying arterial plaque, the method comprising:
analyzing arterial plaque using one or more non-invasive tests to determine if the plaque has any of a plurality of hallmarks that are predictive of disruption, the one or more tests testing the plaque for the plurality of the hallmarks; and
formulating a vulnerability quantity as a function of the determined hallmarks, the vulnerability quantity identifying the degree to which the plaque is vulnerable to disruption.

2. The method as defined by claim 1 further wherein the plaque comprises a plurality of plaque sites along a blood vessel, the method comprising imaging the blood vessel to locate the plurality of plaque sites, each plaque site having an independently determined vulnerability quantity.

3. The method as defined by claim 1 wherein analyzing comprises:
imaging a section of a blood vessel after exposure of that section to a contrast agent; and
quantifying contrast agent absorption of the imaged section.

4. The method as defined by claim 1 wherein analyzing comprises calculating a remodeling ratio of an artery having the plaque.

5. The method as defined by claim 1 wherein analyzing comprises quantifying the heterogeneity of an artery having the plaque.

6. The method as defined by claim 1 wherein analyzing comprises a sequence of hallmark tests that can be performed in a single imaging session.

7. The method as defined by claim 6 wherein analyzing comprises, in the following order:
A. quantifying the heterogeneity of a portion of an artery having the plaque,
B. determining remodeling of the portion of the artery, and then
C. analyzing the circumferential rim of the portion of the artery.

8. The method as defined by claim 1 further comprising converting the vulnerability quantity into a rupture percentage indicating the likelihood of rupture of the plaque.

9. A computer program product for use on a computer system for identifying arterial plaque, the computer program product comprising a tangible computer usable medium having computer readable program code thereon, the computer readable program code comprising:
program code for analyzing arterial plaque received from one or more non-invasive tests that determine if the plaque has any of a plurality of hallmarks predictive of disruption, the one or more tests testing the plaque for the plurality of the hallmarks; and
program code for formulating a vulnerability quantity as a function of the determined hallmarks, the vulnerability quantity identifying whether the plaque is vulnerable to disruption.

10. The computer program product as defined by claim 9 further wherein the plaque comprises a plurality of plaque sites along a blood vessel, the apparatus further comprising program code for imaging the blood vessel to locate the plurality of plaque sites, each plaque site having an independently determined vulnerability quantity.

11. The computer program product as defined by claim 9 wherein the program code for analyzing comprises:
program code for imaging a section of a blood vessel after exposure of that section to a contrast agent; and
program code for quantifying contrast agent absorption of the imaged section.

12. The computer program product as defined by claim 9 wherein the program code for analyzing comprises program code for calculating a remodeling ratio of an artery having the plaque.

13. The computer program product as defined by claim 9 wherein the program code for analyzing comprises program code for quantifying the heterogeneity of an artery having the plaque.

14. The computer program product as defined by claim 9 further comprising program code for converting the vulnerability quantity into a rupture percentage indicating the likelihood of rupture of the plaque.

15. An apparatus for identifying arterial plaque, the apparatus comprising:
an imaging device for non-invasively imaging a blood vessel;
an analysis module operatively coupled with the imaging device, the analysis module configured to analyze arterial plaque imaged by the imaging device using one or more tests to determine if the plaque has any of a plurality of hallmarks that are predictive of disruption, the one or more tests being configured to detect the plurality of the hallmarks; and
a processing module operatively coupled with the analysis module, the processing module formulating an vulnerability quantity as a function of the determined hallmarks, the vulnerability quantity identifying whether the plaque is vulnerable to disruption.

16. The apparatus as defined by claim 15 wherein the analysis module is configured to quantify contrast agent absorption of the imaged section.

17. The apparatus as defined by claim 15 wherein the analysis module is configured to calculate a remodeling ratio of an artery having the plaque.

18. The apparatus as defined by claim 15 wherein the analysis module is configured to quantify the heterogeneity of an artery having the plaque.

19. The apparatus as defined by claim 15 wherein the analysis module is configured to execute a sequence of hallmark tests that can be performed in a single imaging session.

20. The apparatus as defined by claim 15 wherein the analysis module is configured to sequentially execute the following acts:
A. first quantify the heterogeneity of a portion of an artery having the plaque,
B. determine remodeling of the portion of the artery, and then
C. analyze the circumferential rim of the portion of the artery.

21. The apparatus as defined by claim 15 wherein the process module is configured to convert the vulnerability quantity into a rupture percentage indicating the likelihood of rupture of the plaque.
